(19) United States
(12) Patent Application Publication
(10) Pub. No.: US 2023/0371814 A1
(43) Pub. Date: Nov. 23, 2023

(54) WIRELESS THERANOSTIC SMART CONTACT LENS CAPABLE OF MEASURING AND ADJUSTING INTRAOCULAR PRESSURE IN GLAUCOMA PATIENTS

(71) Applicant: POSTECH RESEARCH AND BUSINESS DEVELOPMENT FOUNDATION, Pohang-si (KR)

(72) Inventors: Sei Kwang HAHN, Seoul (KR); Tae Yeon KIM, Pohang-si (KR)

(21) Appl. No.: 18/197,795

(22) Filed: May 16, 2023

(30) Foreign Application Priority Data
May 18, 2022 (KR) 10-2022-0060731
Jan. 2, 2023 (KR) 10-2023-0000164

(51) Int. Cl. A61B 3/16; A61B 3/107; A61F 9/00; G02C 7/04; B29D 11/00

(52) U.S. Cl. CPC A61B 3/16; A61B 3/107; A61F 9/0017; G02C 7/04; B29D 11/00096; A61B 2562/0285; A61B 2017/00022

(57) ABSTRACT

The present invention relates to a wireless theranostic contact lens, which includes gold hollow nanowires having excellent safety and stability in vivo and excellent sensitivity and a controlled drug delivery system allowed to contain a high content of a drug for treating glaucoma.

The contact lens of the present invention is allowed to monitor intraocular pressure in real time and release an appropriate drug from the controlled drug delivery system according to a state of the intraocular pressure, thereby enabling personalized intraocular pressure adjustment.

### CROSS-REFERENCE TO RELATED APPLICATION

This application claims priority to and the benefit of Korean Patent Application No. 2022-0060731, filed on May 18, 2022, the disclosure of which is incorporated herein by reference in its entirety.

### BACKGROUND

### 1. Field of the Invention

The present invention relates to a wireless theranostic smart contact lens capable of measuring and adjusting intraocular pressure in glaucoma patients.

### 2. Discussion of Related Art

Nanowires are widely used in the manufacture of stretchable and transparent electronic materials. However, silver-based nanowires have a problem that biocompatibility and stability are low due to elution of silver ions in vivo. In addition, nanowires have excellent stretchability due to their structure and many studies on the manufacture of stretching strain sensors have been conducted, whereas there is a problem in manufacturing and using the nanowires as sensors for low strain such as intraocular pressure due to their low sensitivity.

A controlled drug delivery system has an advantage of maximizing therapeutic effects of drugs because the system can selectively release the drug by various external stimuli. In particular, a drug delivery system controlled by electrical signals has an advantage of having high controllability of release. However, since the content of the drug may be lowered in a narrow space such as a contact lens, there is a limitation in using the controlled drug delivery system for actual treatment for medical purposes.

Glaucoma is one major cause of blindness and is a chronic eye disease that cannot be cured. Intraocular pressure is regarded as one of the most important indicators that can indicate the degree of disease progression in glaucoma patients. Methods of using the Goldmann applanation tonometer, rebound tonometer, and non-contact tonometer, which are existing methods of measuring intraocular pressure, have a limitation that these methods can only measure static intraocular pressure, and have a problem that errors may vary depending on an environment or person to be measured. In particular, it is impossible to accurately monitor a state of intraocular pressure of glaucoma patients because it is impossible to continuously measure the intraocular pressure, and thus, recently, studies on a system for continuously monitoring intraocular pressure using a contact lens have been actively conducted.

Eye drops are the most basic drug treatment for treating eye diseases and are the primary treatment method for glaucoma patients. However, eye drops have a limitation in that bioavailability is low because many amounts of the drug are washed out by tears or repeated blinking. Drug delivery using smart contact lenses can alleviate the washout of the drug and allow the drug to stay in the eye for a long period of time, resulting in high bioavailability. Recently, studies have been published showing that drug delivery using smart contact lenses has a high therapeutic effect on many eye diseases, and many related studies are being conducted. However, there is a limitation in that release of the drug cannot be precisely controlled.

Recently, many studies have been conducted on smart contact lenses for continuous intraocular pressure monitoring and drug delivery for glaucoma diagnosis. However, intraocular pressure sensors have limitations of low sensitivity, low biocompatibility, low transparency, and low stretchability. In the case of drug delivery, there are limitations for a low loading amount of drug, absence of release control system, and low flexibility. In addition, there is no glaucoma feedback system that monitors a state of intraocular pressure and releases the drug appropriately according to the intraocular pressure, and there is no system in which a glaucoma feedback system is integrated into a contact lens. An integrated theranostic smart contact lens can maximize therapeutic effects of the drug, minimize side effects, and can be used as personalized treatment by releasing the drug appropriately according to the patient's condition.

### SUMMARY OF THE INVENTION

The present invention is directed to providing gold hollow nanowires having excellent safety and stability in vivo and excellent sensitivity.

The present invention is also directed to providing a contact lens, which includes gold hollow nanowires and a controlled drug delivery system that can contain a high content of a drug for treating glaucoma.

According to an aspect of the present invention, there is provided a contact lens for measuring intraocular pressure or treating glaucoma in glaucoma patients, which includes an intraocular pressure sensor and a drug reservoir, wherein the intraocular pressure sensor includes gold hollow nanowires and measures a change in curvature of an eyeball caused by a change in intraocular pressure.

According to another aspect of the present invention, there is provided a method of manufacturing the contact lens for measuring intraocular pressure or treating glaucoma in glaucoma patients, which includes forming a water-soluble sacrificial layer on a handling substrate, forming a transparent substrate on the sacrificial layer, forming an intraocular pressure sensor and a drug reservoir on the transparent substrate, and transferring the transparent substrate on which the intraocular pressure sensor and the drug reservoir are formed into the contact lens.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1A shows a structure of a contact lens, FIGS. 1B and 1C show a drug reservoir and a sensor, and FIG. 1D shows a set of schematic diagrams obtained by measuring and adjusting intraocular pressure;

FIG. 2 shows synthesis analysis data of gold hollow nanowires (AuHNWs), and specifically, FIG. 2A shows a schematic diagram of an Ag@Au core-shell nanowire (Ag@AuNW) and an AuHNW, FIGS. 2B and 2C show a transmission electron microscopy (TEM) image and electron energy loss spectroscopy (EELS) images of AuHNWs, respectively (scale bars: 1 μm and 100 nm, respectively), FIG. 2D shows normalized absorbances of Ag nanowires (AgNWs), Ag@AuNWs, and AuHNWs, FIG. 2E shows optical microscope (OM) images (scale bar: 5 μm) of Ag@AuNWs and AuHNWs films, FIG. 2F shows changes in average transmittance of each nanowires with increasing coating time, FIGS. 2G and 2H show electromechanical properties (n (=6) independent experiments) and electro-thermal properties (n (=3) independent experiments) of Ag@AuNWs and AuHNWs, FIG. 2I shows chemical stability test data and shows changes in relative resistance of nanowires and a gold thin film when exposed to H₂O₂ (30%), FIG. 2J shows changes in relative resistance of AuHNWs and Ag@AuNWs at high pressure (strain, 200 mmHg), and FIG. 2K shows changes in resistance of AuHNWs at low pressure;

FIG. 3 shows AuHNW-based intraocular pressure sensor analysis data, and specifically, FIG. 3A shows OM images (scale bar: 200 μm) of a patterned AuHNW-based intraocular pressure sensor, FIG. 3B shows a photograph (scale bar: 6 mm) of a contact lens in which an intraocular pressure sensor is embedded, FIG. 3C shows an OM image (scale bar: 600 μm) of an intraocular pressure sensor and a radio circuit, FIGS. 3D and 3E show intraocular pressure measurement sensitivity analysis and show changes in relative resistance of an intraocular pressure sensor with different substrate thicknesses (n (=3) independent experiments) and coating time and design (n (=3) independent experiments), respectively, and FIG. 3F shows results of repeatedly measuring intraocular pressure;

FIG. 4 shows analysis data of a controlled drug delivery system, and specifically, FIGS. 4A and 4B show contact lenses in which drug delivery systems for daily and weekly use are embedded, respectively, (scale bar: 5.5 mm), FIGS. 4C and 4D show drug reservoirs that are before and after being opened (selective electrochemical dissolution of gold channels) by an electrical signal, respectively (scale bar: 400 μm), FIG. 4E shows results of electrochemical analysis of the controlled drug delivery system under different bending conditions, FIG. 4F shows drug release efficiency analysis experiment data in the drug delivery system (n (=3) independent experiments), FIG. 4G shows an in vitro release profile from a drug reservoir, and FIG. 4H shows a cumulative drug release profile from three drug reservoirs (scale bar: 5.5 mm).

FIG. 5 shows results of biostability analysis of a contact lens, and specifically, FIG. 5A shows fluorescence microscopy images of the NIH 3T3 cells after live/dead analysis (scale bar: 200 μm), FIG. 5B shows relative cell viability of each sample (n (=3) independent experiments), FIG. 5C shows results of analysis of corneal damage in rabbit's eyes, FIGS. 5D and 5E show OM images and results of cornea thickness analysis of glaucoma-induced rabbit corneas, respectively, (n (=3) independent experiments), and FIG. 5F shows a photographic image (left) for wireless power transfer and communication and a photographic image (right) for thermal characterization of a contact lens for a rabbit's eye with a control board;

FIG. 6 shows intraocular pressure measurement and adjustment analysis data of a contact lens, and specifically, FIG. 6A shows an image of a contact lens mounted on a rabbit's eye (scale bar: 5.5 mm), FIG. 6B shows results of analysis of a correlation between a contact lens and a commercial tonometer, FIG. 6C shows continuous intraocular pressure measurement data with the contact lens and the tonometer (n (=3) independent experiments with tonometer), FIG. 6D shows evaluation results of intraocular pressure lowering ability by timolol released from the contact lens (n (=25) independent experiments), FIG. 6E shows results of intraocular pressure monitoring and intraocular pressure control of the contact lens by timolol release, and FIG. 6F shows results of Bland-Altmann analysis after comparing intraocular pressures measured by a conventional commercial tonometer and a smart contact lens; and

FIG. 7 shows results of glaucoma-related biomarker analysis in a glaucoma-induced rabbit's retina using evaluation results of glaucoma therapeutic ability of contact lenses. Specifically, FIG. 7A shows retinal tissue analysis, FIG. 7B shows glial fibrillary acidic protein (GFAP), FIG. 7C shows CD11b, FIG. 7D shows brain-derived neurotrophic factor (BDNF), and FIG. 7E shows expression results of a Brn3a biomarker.

### DETAILED DESCRIPTION OF EXEMPLARY EMBODIMENTS

The present invention relates to a contact lens for measuring intraocular pressure (IOP) or treating glaucoma in glaucoma patients, which includes an IOP sensor and a drug reservoir.

In the present invention, a contact lens is provided in which an IOP sensor based on gold hollow nanowires (AuHNWs) with excellent transparency, stretchability, biocompatibility, and sensitivity, a drug delivery system (DDS) including high-content therapeutic drug, an application-specific integrated circuit (ASIC) chip for wireless driving and communication, and an antenna are integrated. The contact lens of the present invention is a new type of feedback system that has improved the problems of low stretchability, biocompatibility, sensitivity, and drug content, which are problems of existing DDSs for diagnosis and treatment. In embodiments of the present invention, the possibility of application in the medical field was confirmed by confirming the IOP treatment ability through accurate IOP measurement and drug release using a glaucoma-induced rabbit.

Hereinafter, the present invention will be described in more detail.

In the present invention, the contact lens for measuring IOP or treating glaucoma in glaucoma patients may be expressed as a wireless theranostic smart contact lens.

The contact lens of the present invention may be based on one or more selected from the group consisting of an elastomer such as a silicone elastomer, silicone hydrogel, polydimethyloxane (PDMS), poly(2-hydroxyethylmethacrylate) (PHEMA), and poly(ethylene glycol) methacrylate (PEGMA).

In the present invention, a transparent substrate is formed inside the contact lens, and an IOP sensor and a drug reservoir are formed on the transparent substrate.

The transparent substrate has excellent light transmittance, excellent flexibility and elasticity, and excellent biocompatibility. The transparent substrate may include one or more selected from the group consisting of parylene C PDMS, a silicone elastomer, polyethylene terephthalate (PET), and polyimide (PI).

A thickness of the transparent substrate may affect sensitivity of the IOP sensor, and the sensitivity according to the IOP may increase as the thickness decreases.

In the present invention, the IOP sensor is a transparent sensor that measures IOP of an object, and may measure a change in curvature of an eyeball caused by a change in IOP.

In one embodiment, the IOP sensor may include an AuHNW layer formed on the transparent substrate, a D-poly(3,4-ethylenedioxythiophene) (D-PEDOT) layer formed on the AuHNW layer, and a passivation layer formed on the D-PEDOT layer. In this case, the IOP sensor may be formed on a surface toward the eyeball on the transparent substrate.

In one embodiment, AuHNWs are nanowires (NWs) composed of a hollow core and a gold shell, and may serve as a sensor. In the present invention, due to the hollow structure, the AuHNWs may have various optical properties. In particular, the gold-based NWs may have high absorbance in a visible light region, and accordingly, high transparency may be secured. Further, since the AuHNWs have a hollow structure of a thin gold, the AuHNWs may have high sensitivity in electro-mechanical properties unlike conventional NWs, and has an advantage of being less sensitive to external stimuli.

In the AuHNWs, a thickness of the shell may range from 1 to 100 nm, 10 to 50 nm, or 20 to 30 nm.

In one embodiment, the AuHNW layer may be formed by spin-coating the AuHNWs, and patterned as a double line, and thus an IOP sensor having high sensitivity may be manufactured.

In one embodiment, the D-PEDOT layer includes PEDOT:PSS(poly(3,4-ethylenedioxythiophene):poly(styrenesulfonate)). The D-PEDOT layer may form an electrical path between the AuHNWs to increase electrical conductivity of the sensor and increase safety of the AuHNWs. The D-PEDOT layer may additionally include D-sorbitol. Due to such the D-sorbitol, electrical conductivity and stretchability of PEDOT:PSS can be improved. Accordingly, the IOP sensor may have excellent electrical conductivity while having a fine pattern structure.

Further, the passivation layer may include a component having excellent elasticity and flexibility, and biocompatibility, and specifically, may include one or more selected from the group consisting of thermoplastic polyurethane (TPU), parylene C PDMS, a silicone elastomer, PET, and PI.

In one embodiment, a structure of the pressure sensor is not particularly limited and the pressure sensor may have a circular structure or a straight-line structure. Specifically, the pressure sensor may have a structure that entirely or partially surrounds a cornea of an eyeball. Further, the IOP sensor may be manufactured in a circular design in order to monitor a change in resistance caused by radial deformation of the cornea according to an increase in IOP.

In the present invention, the drug reservoir may be expressed as a DDS. The drug reservoir may be sealed by an electrode pattern containing gold and be in conjunction with the pressure sensor described above, and when an abnormality in the change in IOP is detected by the pressure sensor, the gold of the electrode pattern in the drug reservoir may be dissolved in chlorine in the living body, and the drug may be released from the drug reservoir.

In one embodiment, the drug reservoir may include an electrode pattern containing gold formed on a portion of a surface of the transparent substrate, and a drug well layer formed on the electrode pattern and including one or more drug wells formed to be inserted toward the outside. In this case, holes may be formed in the transparent substrate, and the electrode pattern may surround the holes.

In one embodiment, the drug may be positioned in the drug well, and the drug may be drug capable of treating glaucoma, for example, timolol.

In one embodiment, the drug may be made in the form of powder. In order to apply the drug reservoir in glaucoma treatment, a sufficient amount of the drug should be supported in a limited area called a drug reservoir used in the contact lens. In the present invention, in order to increase a supported amount of the drug, the drug may be supported in the form of powder.

In one embodiment, a plurality of drug reservoirs may be included in one contact lens for daily use as well as weekly use, and specifically, 14 or fewer drug reservoirs, 10 or fewer drug reservoirs, or 7 or fewer drug reservoirs may be included.

The amount of the drug supported in one drug reservoir may be an amount corresponding to the efficacy of one drop of commercially available eye drops.

In one embodiment, a protection layer of a biodegradable polymer may be formed on the drug. A sealing layer formed on the supported drug may make dissolution of the drug difficult because a raw material of the sealing layer penetrates into the powder. In the present invention, in order to solve the above problem, the protection layer may be formed, the drug may be protected through the protection layer, and release efficiency of the drug can be improved. Polyvinyl alcohol (PVA) may be used as the biodegradable polymer.

In the present invention, in addition to the above-described pressure sensor and drug reservoir, an antenna may be additionally formed on the transparent substrate. The antenna may be formed on the transparent substrate to be on the same surface as the sensor.

The antenna may transmit or receive power and signals to or from the outside through induced current and electromagnetic resonance.

In one embodiment, the antenna may be a circular antenna having a circular structure.

In one embodiment, the antenna may be composed of nanomaterials, and the nanomaterials may include one or more selected from the group consisting of zero-dimensional materials such as nanoparticles, one-dimensional nanomaterials such as NWs, nanofibers, or nanotubes, and two-dimensional nanomaterials such as graphene, MoS2, or nanoflakes.

Both the pressure sensor and the antenna may be composed of nanomaterials, and the pressure sensor and the antenna may serve as a pressure sensor and an antenna, respectively, due to differences in pattern structure and nanomaterial content. For example, the antenna may include silver NWs, and may be formed to have a thickness greater than that of the pressure sensor, and by varying an amount of the nanomaterials and the length of the nanowire, and thus it is possible to prevent resistance from being changed according to the change in IOP.

Further, in the present invention, an ASIC chip or the like may be formed on the transparent substrate.

Further, the present invention relates to a method of manufacturing the contact lens for measuring IOP or treating glaucoma in glaucoma patients described above, that is, a method of manufacturing a contact lens.

The method of manufacturing the contact lens may include an operation S1 of forming a water-soluble sacrificial layer on a handling substrate, an operation S2 of forming a transparent substrate on the sacrificial layer, an operation S3 of forming a pressure sensor and a drug reservoir on the transparent substrate, and an operation S4 of transferring the transparent substrate on which the pressure sensor and the drug reservoir are formed into the contact lens.

The operation S1 is an operation of forming the sacrificial layer on the handling substrate.

The sacrificial layer may serve as an adhesive layer between the handling substrate and the transparent substrate, and help transfer of the transparent substrate on which the pressure sensor and the drug reservoir are formed. The sacrificial layer is not particularly limited as long as it is soluble in water, and may include one or more selected from the group consisting of PVA and dextran.

The operation S2 is an operation of forming the transparent substrate on the sacrificial layer, wherein the sacrificial layer serves as an adhesive, and thus the transparent substrate may be easily attached to the handling substrate, and may be easily separated from the handling substrate through dissolution of the sacrificial layer in a later process.

In one embodiment, a material having excellent light transmittance may be used as the transparent substrate, and the above-described type of material may be used as the transparent substrate.

The operation S3 is an operation of forming the pressure sensor and the drug reservoir on the transparent substrate.

In one embodiment, the pressure sensor may be manufactured through an operation a1 of forming a mask material for patterning on the transparent substrate, an operation a2 of coating AuHNWs on the transparent substrate on which the mask material is formed through a lift-off process and forming an AuHNW layer, an operation a3 of forming a D-PEDOT layer on the AuHNW layer, and an operation a4 of forming a passivation layer on the D-PEDOT layer.

The operation a1 is an operation of forming the mask material for patterning on the transparent substrate.

The mask material may serve as a shadow mask, and the AuHNWs may be patterned using the mask material. A material that can be used as a photoresist may be used as the mask material, and specifically, SU-8 may be used as the mask material.

The operation a2 is an operation of coating the AuHNWs on the transparent substrate on which the mask material is formed through the lift-off process and forming the AuHNW layer.

Through this operation, a pattern of the AuHNWs may be formed.

In one embodiment, the AuHNWs are NWs composed of a hollow core, and a gold shell. The AuHNWs may be prepared by growing gold on a surface of the NWs to synthesis Ag@Au core-shell NWs (Ag@AuNWs) and then selectively fusing silver in a central portion (core).

The AuHNWs prepared in this operation may act as a pressure sensor. The pressure sensor is made of AuHNWs, and may measure IOP through a change in current and a change in resistance of the sensor according to a change in curvature of the eyeball using connection and disconnection between the AuHNWs.

Further, in this operation, a circuit may be patterned, and the patterned circuit may serve to connect the pressure sensor, the antenna, and the drug reservoir.

The operation a3 is an operation of forming the D-PEDOT layer on the AuHNW layer.

In this operation, the D-PEDOT layer may be formed by coating a D-PEDOT solution on the AuHNW layer. The D-PEDOT solution may be prepared by adding D-sorbitol to a PEDOT:PSS solution.

The operation a4 is an operation of forming the passivation layer.

In this operation, the passivation layer may be formed to prevent the AuHNWs from being lost, and thus electrical stability can be improved. The passivation layer may include the above-described type of component.

In one embodiment, the drug reservoir may be prepared through an operation b1 of forming an electrode pattern containing gold on a portion of a surface of the transparent substrate, an operation b2 of forming a drug well layer including one or more drug wells on the electrode pattern, an operation b3 of supporting the drug into the drug well, and an operation b4 of forming a protection layer on the drug.

The operation b1 is an operation of forming the electrode pattern containing gold on a portion of a surface of the transparent substrate.

In the present invention, the electrode pattern may be manufactured using two methods. In a first method, one or more holes may be formed in the transparent substrate on which the drug reservoir is formed, and the electrode pattern may be formed to surround the holes. Specifically, the substrate in which the holes are formed may be coated with a polymer such as PVA or the like, and electrodes may be deposited on the polymer and then patterned in a desired shape.

In a second method, the electrode pattern may be formed on the transparent substrate, and then holes may be formed in an opposite surface using a laser, specifically, a carbon dioxide laser.

In one embodiment, the electrode pattern stacked on the transparent substrate may include an anode made of a metal containing gold, and a cathode commonly connected to the anode, and in this case, the gold of the electrode pattern may act as a drug release channel. The electrode may have a form in which a plurality of anodes form an array. The electrode may be made of gold and titanium according to a portion. The gold forming the electrode pattern may be electrolyzed and removed by a voltage applied into an electrolyte. Therefore, the anode of the electrode pattern may be used as a gate (drug release channel) of a path through which the accommodated drug is delivered by the voltage.

The operation b2 is an operation of forming the drug well layer including one or more drug wells on the electrode pattern. The drug may be stored in the drug well.

The drug well layer including the drug wells may include a flexible and biocompatible component, and specifically, may include one or more selected from the group consisting of SU-8, PDMS, a silicone elastomer, and polyurethane acrylate (PUA).

In the present invention, the transparent substrate including the drug reservoir may be manufactured by separately preparing the drug well layer and then forming the drug well layer on the electrode pattern.

The operation b3 is an operation of supporting the drug into the drug well.

The drug may be a drug for glaucoma treatment, and specifically, timolol. The drug may be supported in the form of powder and a supported amount of the drug may be maximized.

The operation b4 is an operation of forming the protection layer on the drug. The sealing layer formed on the supported drug may make dissolution of the drug difficult because a raw material of the sealing layer penetrates into the powder. In the present invention, in order to solve the above problem, the protection layer may be formed to protect the drug. PVA may be used as the biodegradable polymer.

In the present invention, the sealing layer may be formed on the drug well on which the drug is supported.

Further, the operation S4 is an operation of transferring the transparent substrate on which the pressure sensor and the drug reservoir are formed into the contact lens.

The sensor and the drug reservoir manufactured on the sacrificial layer may be transferred while dissolving the sacrificial layer in biocompatible water.

Further, the present invention may further include an operation of forming an antenna on the transparent substrate. The operation may be performed in operation S3, and may be performed in the same way as the method of manufacturing the pressure sensor.

Further, the present invention relates to a wireless driving system for measuring IOP or treating glaucoma in glaucoma patients.

The wireless driving system of the present invention may include a contact lens and smart glasses, which include a transparent pressure sensor for measuring IOP of an object and a drug reservoir.

The above-described contact lens may be used as the contact lens. Specifically, in the contact lens, a pressure sensor and a drug reservoir are formed on a transparent substrate. In addition, the pressure sensor may measure a change in current or a change in resistance caused by a change in IOP, and when an abnormality in the change in IOP is detected, drug may be released from the drug reservoir.

In one embodiment, the pressure sensor and the drug reservoir may be connected to an ASIC chip to enable wireless communication. The pressure sensor and the drug reservoir may be driven by receiving an electrical signal transmitted from an external system through the ASIC chip, and may transmit a result detected by the pressure sensor to the external system to store and process data and control the driving of a DDS.

In one embodiment, the drug reservoir may receive the electrical signal transmitted from the external system, whereby the gold of the electrode pattern is dissolved in chlorine in the living body to become AuC14−, and the electrode pattern is opened so that the drug may be released from the drug reservoir to the outside.

In the present invention, the smart glasses may transmit or receive the electrical signal wirelessly to control the deriving of the pressure sensor and the drug reservoir of the wirelessly driven contact lens. In the present invention, it is possible to provide smart glasses capable of adjusting a long distance in micro units using technologies of transparent electrodes using nanomaterials, stretchable electronics, complementary metal-oxide semiconductor (CMOS), flexible and biocompatible micro electro-mechanical system (MEMS), and nano electro-mechanical system (NEMS).

In the smart glasses, electrical power may be demonstrated using wireless inductive power transfer (witricity) technology, and wireless communication can be performed using Bluetooth, infra-red (IR) rays, and radio frequency (RF) in the smart glasses.

A driving system of the smart glasses is an Android operating system (OS), and an open multimedia applications platform (OMAP) 4430 SoC, a dual-core central processing unit (CPU), and a 4 GB random access memory (RAM) may be installed thereon. A display screen is composed of 640*360 pixels, and a bone conduction transducer may be used for sound. The driving system may control functions of an optical sensor, a bio sensor, pressure, temperature, and an acoustic emission (EM) sensor using voice through a microphone, and may be paired with a smartphone, smart watch, or PC. A built-in 100 mAh lithium-ion battery is used as power, and a photocell may be inserted for self-powering. The driving system weighs less than 20 g in total and may be equipped with Wi-Fi 802.11b/g, Bluetooth, and micro-Universal Serial Bus (USB). Photos: >15 MP; and videos: >720p may be possible.

In one embodiment, the sensor may be driven by the electrical signal transmitted from the smart glasses, the sensor that receives the signal may detect a change in current and a change in resistance according to a change in curvature of the eyeball caused by a change in IOP, and the sensor may transmit a result of the detection to the smart glasses through RF wireless communication.

Further, in one embodiment, the DDS may be driven by the electrical signal transmitted from the smart glasses, and as the gold electrode pattern that seals the drug reservoir of the DDS receives the signal, the gold electrode pattern is dissolved in chlorine to become AuC14−, and thus the drug reservoir may be opened.

Further, the present invention relates to a method of measuring IOP or treating glaucoma in glaucoma patients using the wireless driving system described above.

In the treatment method according to the present invention, the pressure sensor in the contact lens may measure a change in current and a change in resistance by applying a voltage to an eyeball of an object at a predetermined measurement period of time, and when it is measured that the change in current and the change in resistance caused by the change in IOP of the eyeball of the object is greater than equal to a set range, the gold of the electrode pattern that seals the drug well of the drug reservoir is dissolved in chlorine to become AuC14−, and thus the drug reservoir may be opened.

In one embodiment, the pressure sensor may be driven by the electrical signal transmitted from the smart glasses, the pressure sensor that receives the signal may measure a change in current and a change in resistance caused by a change in IOP, and the pressure sensor may transmit a result of the change to the smart glasses or the outside through wireless communication.

In one embodiment, the drug reservoir may be driven by the electrical signal transmitted from the smart glasses or the outside, the smart glasses may analyze the change in current and the change in resistance transmitted through the pressure sensor, and when an abnormality of the change in IOP is detected, the smart glasses transmit the electrical signal to the drug reservoir, and the drug reservoir that receives the signal may be opened.

Further, in one embodiment, power generated in the wireless electric coil (witricity coil) of the smart glasses may be received by the wireless electric antenna (witricity antenna) of the wirelessly driven contact lens, and the received power may be used to drive the sensor and the DDS through the control of an integrated circuit (IC) chip.

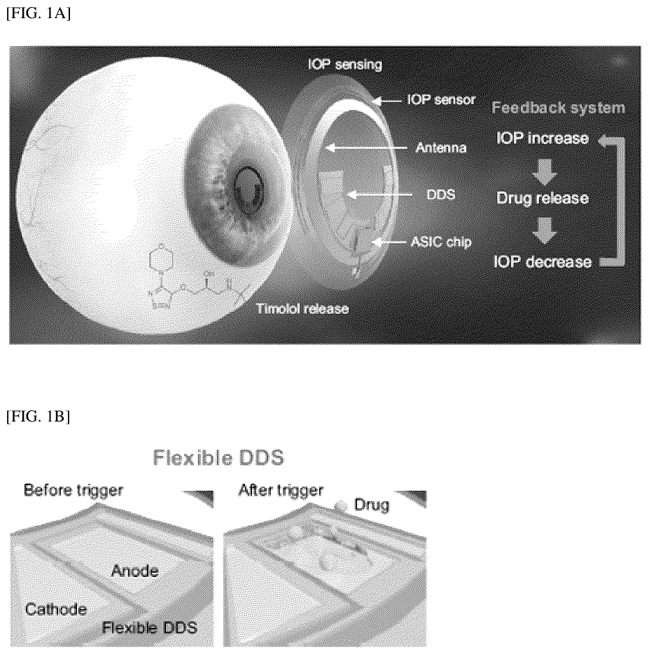

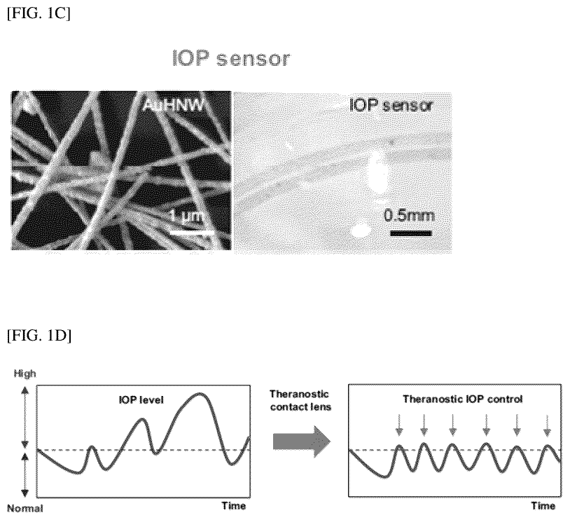

In the present invention, FIGS. 1A to 1D are schematic diagrams of the contact lens according to the present invention for treatment of glaucoma.

As shown in the drawings, it can be seen that the contact lens of the present invention is composed of an AuHNW-based IOP sensor, a DDS, and a radio circuit equipped with a feedback system for detecting IOP and releasing the drug. Further, it is possible to continuously monitor IOP and to control IOP by IOP monitoring and on-demand drug delivery for glaucoma treatment.

Hereinafter, the present invention will be described in detail with reference to the following examples. However, the following examples are merely for providing examples of the present invention, and the content of the present invention is not limited to the following examples.

### EXAMPLES

### Preparation Example 1. Synthesis of Ag@AuNWs

Ag@AuNWs were synthesized by growing gold on a surface of silver NWs.

### Preparation Example 2. Synthesis of AuHNWs for High Transparency and High Sensitivity Sensing

AuHNWs were synthesized by selective etching of an Ag core in 30% dilute nitric acid (SAMCHUN). Specifically, the nitric acid was slowly added to an Ag@AuNW solution with a volume ratio of 1:1. After 1 hour, the AuHNWs were cleaned several times with ethanol and deionized water for further characterization thereof.

### Experimental Example 1. Structural Analysis of AuHNWs

A structure of the AuHNWs prepared in Preparation Example 2 was examined by electron energy loss spectroscopy (EELS) equipped with a high-resolution transmission electron microscope (HRTEM) (JEM-2200FS, JEOL). Further, networks and structures of the AuHNWs and Ag@AuNWs were analyzed using a scanning electron microscope (SEM) (MIRA3, TESCAN).

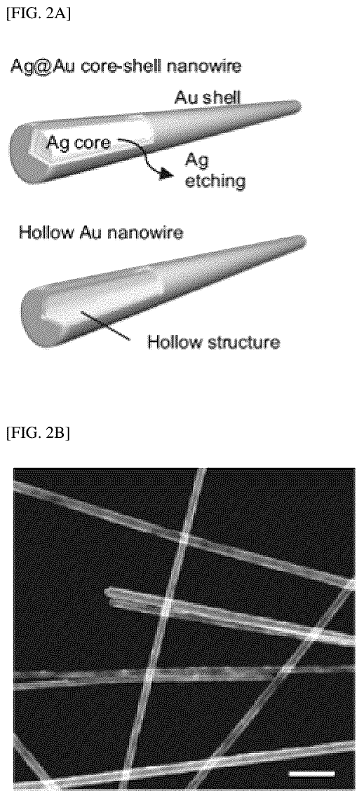

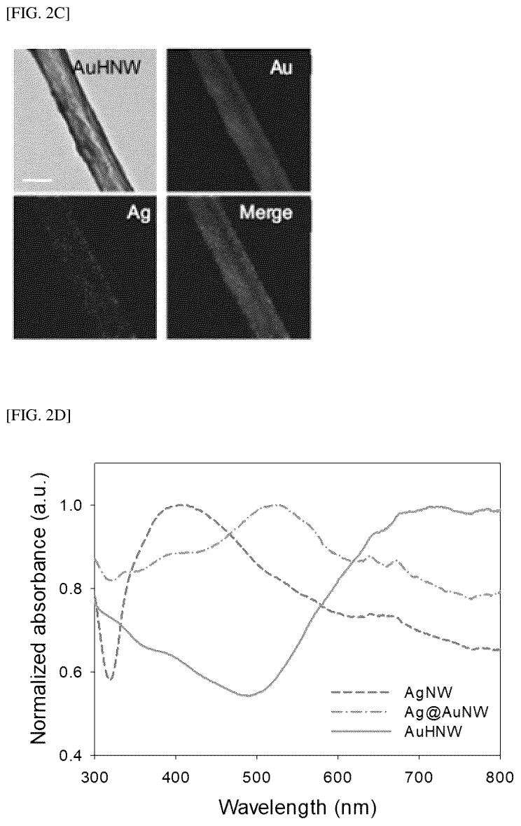

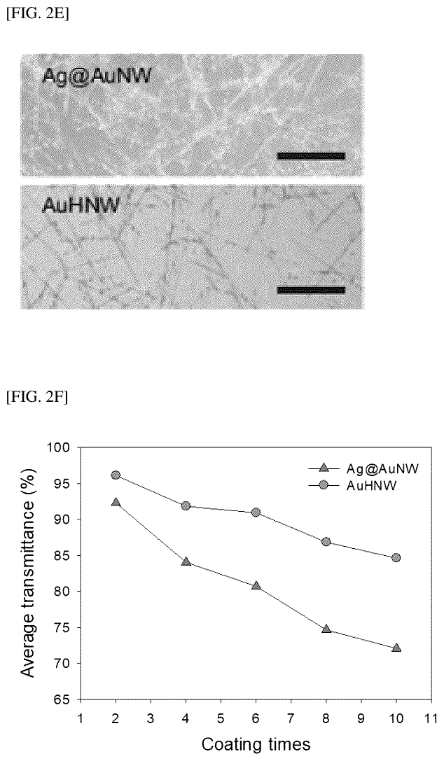

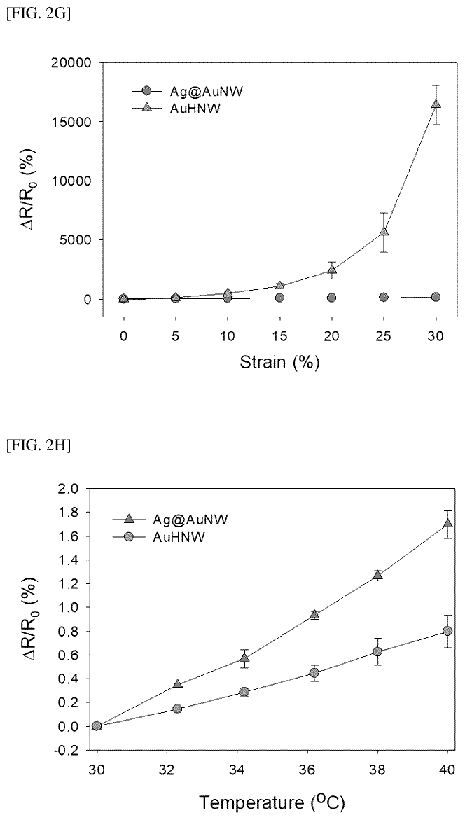

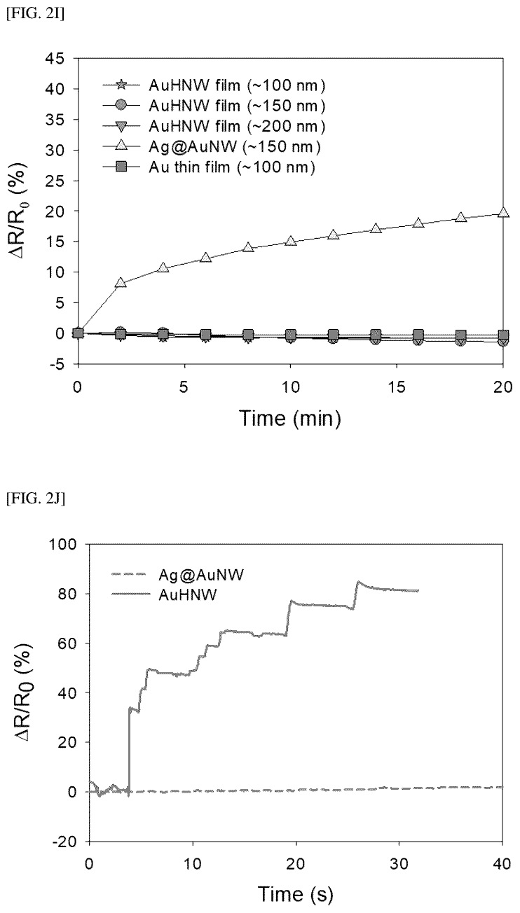

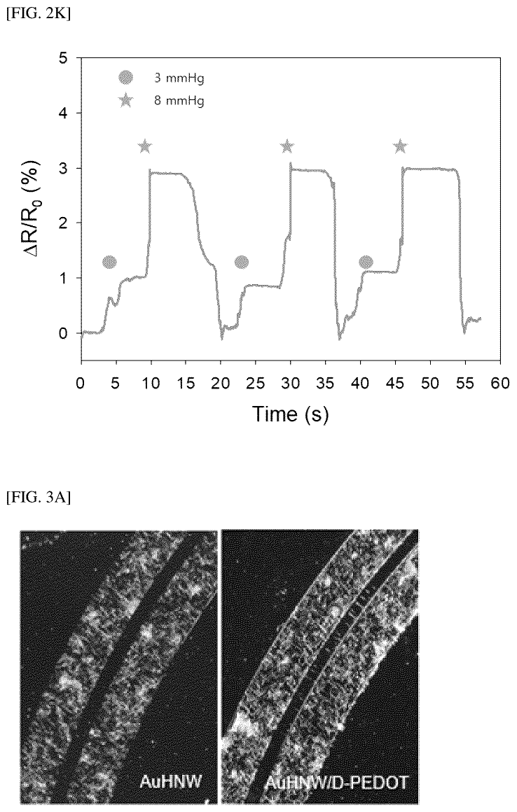

FIGS. 2A to 2C show the structure of AuHNWs. Specifically, FIG. 2A is a set of schematic diagrams of Ag@AuNWs and AuHNWs, and FIGS. 2B and 2C are a transmission electron microscope (TEM) image and EELS images of AuHNWs.

In this example, AuHNWs was prepared by etching an Ag core of the Ag@AuNWs to prepare NWs having high transmittance, sensitivity, and chemical stability. It can be seen through a HRTEM that a hollow structure of the AuHNWs had a shell thickness of about 20 to 30 nm. Further, it can be seen through mapping of Ag and Au distributions that the Ag core of the Ag@AuNWs was selectively etched by diluted nitric acid, but the Au shell was not etched, resulting in the formation of hollow NWs.

### Experimental Example 2. Optical Characterization of AuHNWs

For characterization of optical transmittance, the AuHNWs prepared in Preparation Example 2 and the Ag@AuNWs prepared in Preparation Example 1 were plasma-treated and then spin-coated on a parylene C (LAVIDA 110, Femto Science) substrate. Further, for characterization of absorbance, the NWs prepared in Preparation Examples 1 and 2 were dispersed in ethanol. Optical transmittance and absorbance were measured using an ultraviolet (UV)-vis spectrometer (S-3100, Scinco Co.).

FIGS. 2D to 2F show optical properties of the AuHNWs, and specifically, FIG. 2D shows absorbance, FIG. 2E shows optical microscope (OM) images, and FIG. 2F shows a transparency analysis result.

As shown in the drawings, it can be seen that the AuHNWs prepared in Preparation Example 2 had various optical properties due to the hollow structure.

Specifically, it can be seen that the absorbance in a visible light region was significantly reduced due to a change in surface plasmon resonance, but the gold-based NWs show high absorbance in the visible light region. The OM images show different optical properties of Ag@AuNWs and AuHNWs in the visible light region. AgNW, Ag@AuNW, and AuHNW solutions show different colors due to different optical properties, and an average transmittance of AuHNWs is higher than an average transmittance of Ag@AuNWs in the entire visible light region. This may be because an absorbance peak shifted to a near-infrared (NIR) region and decreased in the visible light region. Due to these properties, it can be seen that the AuHNWs have high transparency.

### Experimental Example 3. Electrical Characterization of AuHNWs

For characterization of electromechanical properties, the NWs prepared in Preparation Examples 1 and 2 were spin-coated on PDMS (sylgard 184, Dow Corning), both ends of a nanowire film were connected to a copper wire with a liquid metal, and a relative resistance was measured using a source meter (Keithley 2450) at a constant voltage of 0.65 V. The nanowire film was stretched with a customized stretching machine and a change in resistance was measured using the source meter. Further, for a chemical stability test, each of nanowire films having different coating times was exposed to H₂O₂ (SAMCHUN) and the relative resistance was measured using the source meter. A thickness of the nanowire film was characterized with a three-dimensional (3D) surface profilometer (Bruker, Billerica). For a stability test in phosphate-buffered saline (PBS) (Tech & Innovation), the nanowire film was immersed in PBS (pH 7) at 37° C. for several days.

FIG. 2G shows electromechanical properties of AuHNWs.

As shown in the drawing, it can be seen that the AuHNWs have a hollow structure of a thin gold shell, and thus the AuHNWs have high sensitivity in electromechanical properties unlike conventional NWs.

Further, FIG. 2H shows electro-thermal properties, and FIG. 2I shows chemical stability test results.

As shown in the drawings, it can be seen that the electrical properties of the AuHNWs changed less than the conventional NWs with respect to a change in temperature. Further, it can be seen that absorbance peaks of the AgNW and Ag@AuNWs were reduced by Ag etching by H₂O₂ after 2 hours, but the AuHNWs did not show any change in absorbance. This case was similar to the case of the gold thin film. That is, it can be seen that the AuHNWs of the present invention had inactive properties with high stability.

Further, FIG. 2J shows changes in relative resistance of AuHNWs and a gold thin film at high pressure (strain, 200 mmHg), and FIG. 2K shows a change in resistance of AuHNWs at low pressure.

As shown in the drawings, it can be seen that the AuHNWs of the present invention stably operated even when a pressure (strain, 200 mmHg) higher than a normal IOP range (14 to 21 mmHg) is applied. Further, it can be seen that the AuHNWs had higher IOP measurement sensitivity than conventional bulk NWs (e.g., Ag@AuNWs). It can be seen that a small pressure range of 3 mmHg may be measured and there is no large hysteresis.

That is, it can be seen that the AuHNWs of the present invention had new properties that are excellent in transparency and sensitive to strain but less sensitive to external stimuli. Accordingly, the possibility of using AuHNWs for long-term sensitive monitoring of IOP can be confirmed.

### Preparation Example 3. Manufacture of Hybrid IOP Sensor

Parylene C (LAVIDA 110, Femto Science) (300 nm) serving as a substrate was deposited on a sacrificial layer.

AZ-nLoF (microchemicals) was spin-coated on the parylene C and was patterned by photolithography through a lift-off process.

First, Ag@AuNWs (1 mg/ml) that was dispersed in a mixed solution of ethanol and deionized water (volume ratio of 2:5) was spin-coated 5 times and annealed at 110° C. for 2 minutes. After the annealing, the nanowire-coated substrate was immersed in nitric acid (30% in DI water) for 10 seconds to etch the Ag core. An AuHNW film was annealed at 110° C. for 2 minutes to completely remove the remaining nitric acid.

Prior to D-PEDOT coating, the AuHNW film was treated with argon plasma (150 W, 2 minutes). For a D-PEDOT solution, D-sorbitol (20 mg/ml, Sigma Aldrich) was added to a PEDOT:PSS solution (PH1000, Clevios) while being stirred for 3 hours. The D-PEDOT solution was spin-coated on the AuHNW film and annealed at 110° C. for 5 minutes. Thereafter, a photoresist was removed with acetone and carefully cleaned with isopropyl alcohol (IPA, SAMCHUN) to prepare a hybrid IOP sensor. TPU (100 mg/ml, 1185A, Elastollan) was finally spin-coated on the hybrid IOP sensor for passivation.

The hybrid IOP sensor was moved by dissolving PVA (363170, Sigma Aldrich) and placed in a contact lens mold. The contact lens mold was filled with a silicone elastomer (MED-6015, Nusil) and cured at 100° C. for 1 hour to prepare a soft contact lens.

In order to monitor a relative resistance of the IOP sensor in vitro, a copper wire was connected to the IOP sensor with silver epoxy (MED-H20E, EPO-TEK) or liquid metal (495425, Sigma Aldrich).

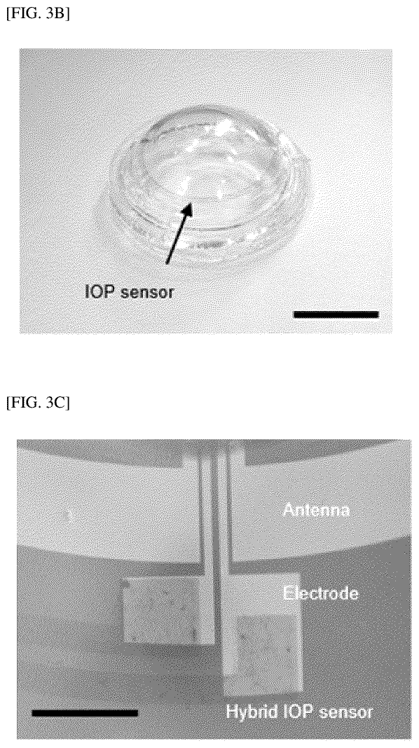

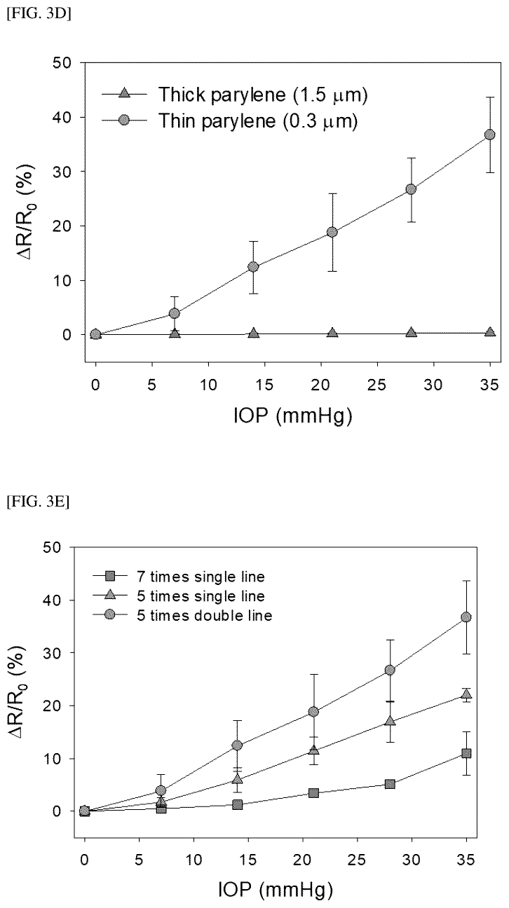

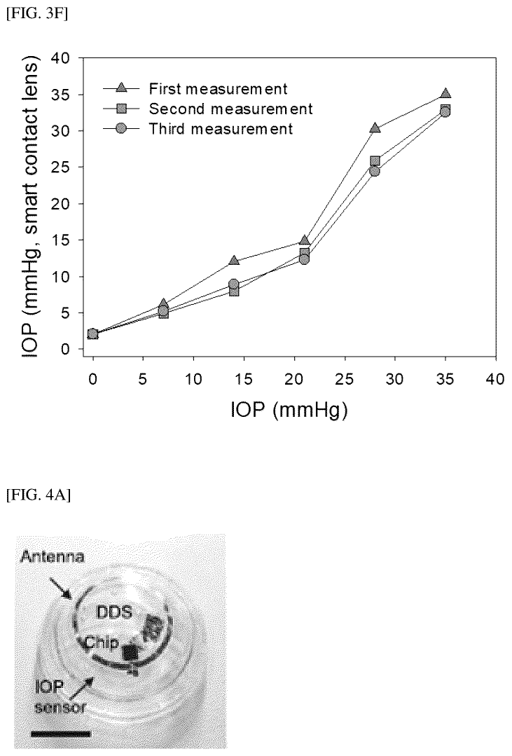

FIG. 3A shows a structure of the IOP sensor manufactured in Preparation Example 3.

As shown in the drawing, the AuHNWs and the D-PEDOT layer may be sequentially coated on the substrate and then patterned through a conventional lift-off process. In the present invention, a pattern of the D-PEDOT layer, that is, a PEDOT:PSS conductive polymer doped with D-sorbitol, was formed, and thus electrical conductivity can be improved and stability of the wire can be improved.

FIGS. 3B and 3C show a photograph of a contact lens having an IOP sensor embedded therein and an OM image of an IOP sensor and a radio circuit.

As shown in the drawings, it can be seen that the IOP sensor embedded and manufactured in the contact lens is very transparent. Further, it can be seen that an AuHNW-based IOP sensor was successfully integrated with an antenna and an electrode.

### Experimental Example 4. Characterization of Hybrid IOP Sensor

An artificial eye model was manufactured using a PDMS mixed base and a curing agent with a weight ratio of 20:115. A diameter of the artificial eye model was 15 mm and a thickness was 500 μm.

The pressure of the artificial eye model was controlled by inserting two scalp vein sets into the artificial eye model. One scalp vein set was connected to a customized pressure sensor, and the other was connected to a syringe pump. After the contact lens manufactured in Preparation Example 3 was mounted on the artificial eye model, the pressure of the artificial eye model was adjusted by injecting or discharging PBS.

A change in resistance of the IOP sensor according to a change in pressure was measured with a source meter at a constant voltage of 0.65 V.

FIGS. 3D and 3E show results of analysis of sensitivity of IOP measurement.

In this experimental example, the thicknesses of the parylene C and AuHNW coating were optimized to improve the sensitivity of the IOP sensor. As shown in the drawings, it can be seen that the thick parylene C had a high elastic modulus and is not easily deformed even when a curvature of a cornea is changed. Although the thin parylene C increases the sensitivity of the IOP sensor, the thin parylene C cannot support the DDS structure. The IOP sensor using the thin parylene C exhibited higher sensitivity than the thick parylene C.

That is, it can be seen that the sensitivity according to the IOP increased as the thickness of the parylene C substrate decreased.

Further, it is possible to manufacture the IOP sensor having high sensitivity by spin-coating the AuHNWs 5 times and patterning the AuHNWs as a double line, that is, by optimizing coating time (concentration) and design.

Further, FIG. 3F shows experimental data of repeated IOP measurements.

As shown in the drawing, it can be seen that there was no significant difference in pattern of a change in relative resistance according to the change in IOP and there is no significant change in measurement capability of the AuHNW-based IOP sensor even when the IOP was repeatedly measured.

### Preparation Example 4. Manufacture of Flexible DDS

Parylene C (300 nm) was deposited on a sacrificial layer. Gold was deposited on the parylene C and patterned to a thickness of 100 nm for a gold channel and 500 nm for an electrode. A drug reservoir was manufactured by spin-coating SU-8 2015 (Kayaku Advanced Materials, Inc) on the gold channel and the electrode. Timolol powder (timolol maleate salt, T6394, Sigma Aldrich) was placed in the reservoir, and a PVA (100 mg/ml) solution serving as a protective layer was coated on the drug and dried at room temperature. An additional parylene C layer (100 nm) was deposited to seal the DDS. The sacrificial layer was melted to transfer the DDS, and a rear surface of the DDS was patterned with a photoresist to selectively etch the parylene C. Opened parylene C was selectively etched with reactive-ion etching (RIE) (O₂, 100 sccm, 150 W, 5 minutes) (Covance, Femto Science).

A flexible DDS was embedded in a contact lens. During a molding process, the gold channel and the cathode were left open to allow the gold to be dissolved by electrochemical reactions in tears. The opened gold channel may be selectively dissolved in PBS by applying a voltage of 1.85 V.

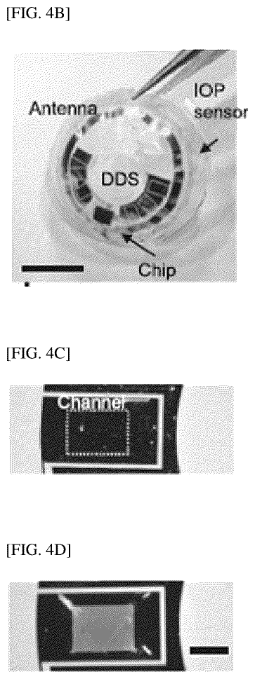

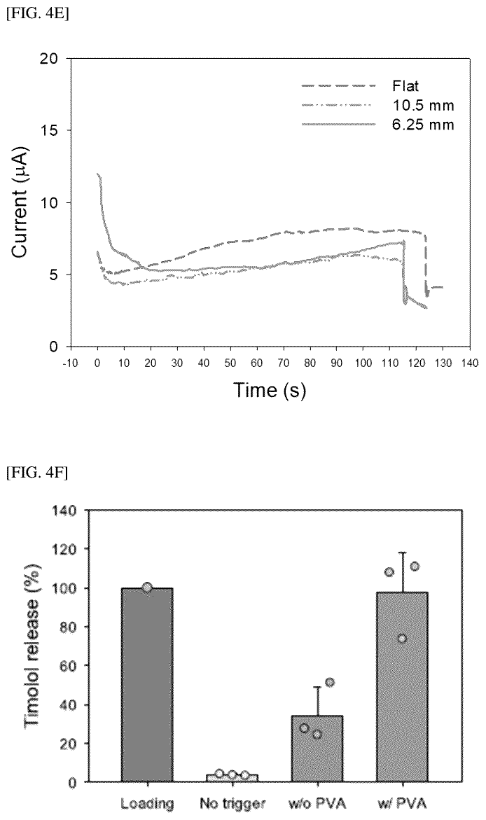

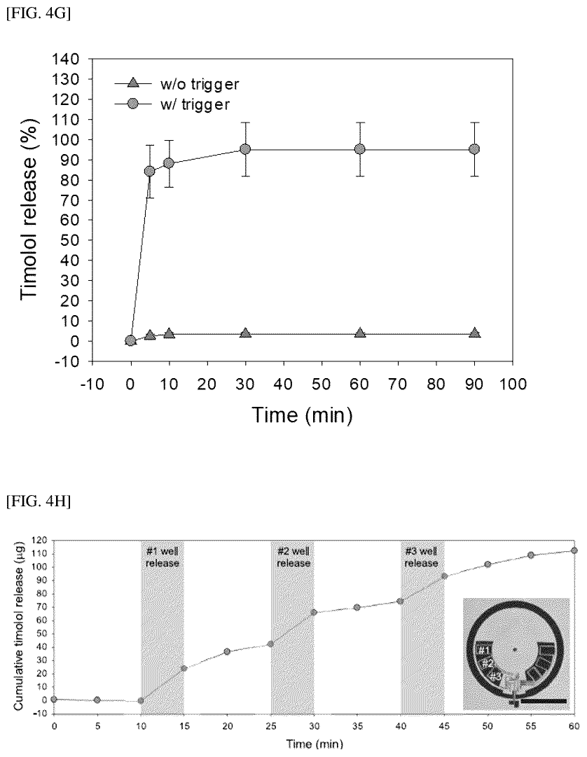

FIG. 4 shows analysis data of a controlled DDS, and specifically, FIGS. 4A and 4B show contact lenses in which DDSs for daily and weekly use and IOP sensors are embedded, respectively.

In this experimental example, two types of DDSs were designed for daily and weekly use.

FIGS. 4C and 4D show opening of a drug reservoir by an electrical signal. In the DDS of the present invention, a gold thin film that surrounds the drug reservoir in which drug is stored is melted by an electrochemical reaction with Cl-ions in vivo, and the drug reservoir may be opened. Accordingly, it is possible to precisely control release of the drug.

### Experimental Example 5. Characteristics of Flexible DDS

An electrochemical reaction of gold was examined in vitro in PBS (pH 7.4) and artificial tears having a constant voltage of 1.85 V.

Timolol was released from the drug reservoir by applying a voltage of 1.85 V to an anode and a cathode of a DDS for 5 minutes. A concentration of the released timolol was quantified with a UV-vis spectrometer at an absorbance wavelength of 259 nm.

FIG. 4E shows results of electrochemical analysis of a DDS (results of current-time (1-t) curve analysis for a period of time during which gold is dissolved).

As shown in the drawing, a dissolution time of the bent DDS was within 140 seconds and an operating current was 6 μA at a curvature of 6.25 mm. It can be seen that the curvature of the contact lens is generally ca. 8 mm and even when the DDS is embedded in the smart contact lens, the contact lens is sufficiently flexible without fatal damage.

That is, it can be seen that the DDS was made of materials having excellent flexibility and stably operated even in a bending state high enough to be inserted into a lens.

FIG. 4F shows experiment data of analysis of drug release efficiency, and FIG. 4G shows an in vitro release profile from the reservoir.

As shown in the drawings, it can be seen that less than 50% of the drug (timolol) was released in the absence of the PVA layer, whereas the drug (timolol) was completely dissolved in the presence of PVA. Further, it can be seen that, in the absence of the PVA layer, less than 5% of timolol was released for 90 minutes without electrical triggering, whereas, in the presence of PVA, less than 85% of timolol was released within 5 minutes and almost all of the timolol was completely released within 30 minutes.

That is, it can be seen that the release efficiency was increased by using the drug in the form of powder for supporting a high-content therapeutic drug and using the PVA that is a biodegradable polymer as a protection layer.

Further, FIG. 4H shows continuous drug release of the controlled DDS.

As a result of examining the cumulative release of timolol by sequentially activating three different reservoirs, one at 10 minutes, 25 minutes, and 40 minutes at 15-minute intervals, it can be seen that there was no significant release of timolol until 10 minutes, but after that, the release of timolol was significantly enhanced at each activation time of the DDS.

That is, it can be seen that the drug can be released through the selective and precise control by continuously releasing several drug reservoirs at regular time intervals.

### Preparation Example 5. Manufacture of Highly Integrated Theranostic Contact Lens

For precise integration of radio circuits, IOP sensors, DDSs, and all other components were sequentially manufactured on a Parylene C substrate.

First, gold with different thicknesses were deposited on the substrate, patterned by photolithography for a DDS electrode (100 nm) and the radio circuit (500 nm) of an antenna and a chip pad, and then an ASIC chip was flip-chip bonded. The IOP sensor was manufactured on the same substrate by a lift-off process in which AuHNWs and D-PEDOT were sequentially coated.

Thereafter, parylene C was selectively etched by RIE to separate the IOP sensor and the radio circuit. SU 8 was used to pattern protection layers for drug reservoirs, chips, antennas, and interconnections. Timolol was loaded into the drug reservoir, and the protection layer of PVA was coated on the loaded drug. TPU was spin-coated on the manufactured theranostic system except for the drug reservoir.

Finally, the parylene C (100 nm) was deposited for passivation and layer sealing. The manufactured theranostic system was transferred by melting the sacrificial layer and embedded in the contact lens through simple molding.

### Experimental Example 6. Cell Viability and Biological Safety Analysis

In order to evaluate cell viability and biosafety of nanomaterials, fibroblasts (NIH 3T3, mouse embryonic fibroblasts) at a concentration of 5.0×10³ cells/ml were directly seeded as blank substrates on parylene C, AuHNWs, and D-PEDOT, and as a control group on AgNWs.

The NIH 3T3 cell line was purchased from the American Type Culture Collection (CRL-1658, ATCC). The concentration of each material was the same as that of the IOP sensor.

Cells from each film were cultured in a cell culture medium, Dulbecco's modified Eagle's medium (DMEM) (Thermo Fisher) for 3 days, and then the cells were stained green and red with Calcein AM and ethidium homodimer-1 (EthD-1) and observed under a fluorescence microscope. A live/dead cell imaging kit was purchased from Thermo Fisher. Cell viability was quantified by counting live cells (green) and dead cells (red) in fluorescent OM images.

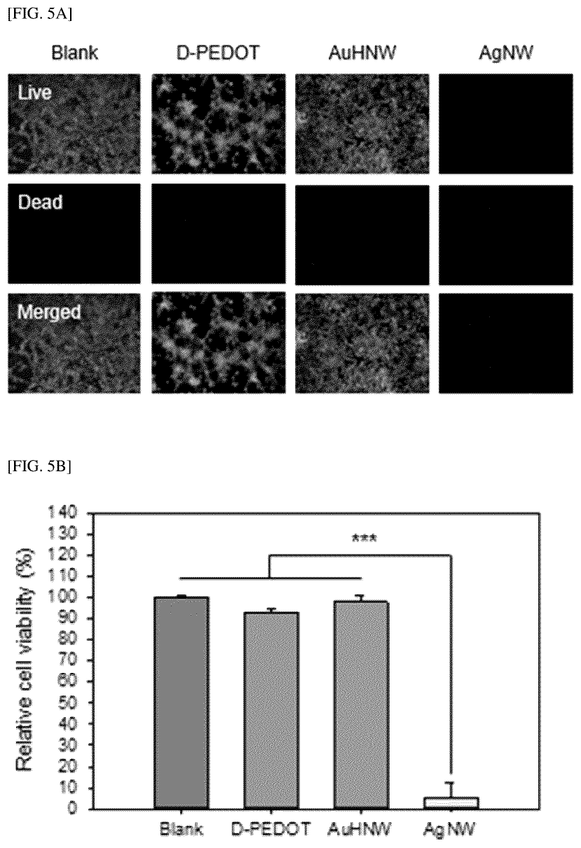

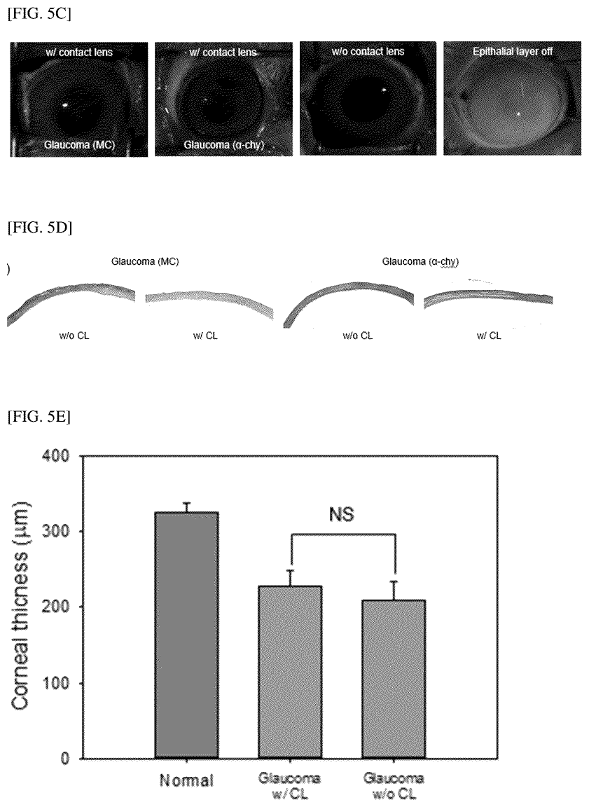

FIG. 5 shows results of biostability analysis of a contact lens, wherein FIGS. 5A and B show results of cytotoxicity test of materials. Specifically, FIG. 5A shows fluorescence microscopy images of NIH 3T3 cells after live/dead analysis, and FIG. 5B shows relative cell viability of each sample.

As shown in the drawings, cells were stained green and dead cells were stained red in the fluorescence optical microscope images. It can be seen that, in the case of silver NWs, most of the cells were killed by released Ag+ ions (<15%), but in the IOP sensor of the present invention, 92% or more of cells survived in all types of materials.

That is, it can be seen that biocompatibility of the nanomaterials used in the contact lens was excellent.

Further, FIG. 5C shows results of damage analysis of a cornea, FIG. 5D shows results of inflammation analysis of the cornea, and FIG. 5E shows results of thickness analysis of the cornea.

As shown in the drawings, it can be seen that corneal damage and inflammation do not appear after the contact lens is worn on a rabbit's eye, and it can be seen that there is no change in thickness of the cornea due to wearing of the lens.

Further, FIG. 5F shows results of heating test of the contact lens.

As shown in the drawing, it can be seen that the analysis of an infrared thermal imaging camera did not show a fatal temperature increase in the contact lens of the rabbit's eye during IOP data collection, wireless communication, and DDS activation by electrical signals.

Accordingly, it is possible to confirm the biostability of the contact lens without fatal damage in glaucoma-induced animal models.

### Experimental Example 7. In Vivo Evaluation of Contact Lens

The contact lenses manufactured in Preparation Example 5 were worn on both eyes of a glaucoma-induced rabbit for 1 hour every other day. After 2 weeks, fluorescence staining analysis of the cornea and cornea thickness analysis were performed to evaluate biosafety.

Specifically, an IOP sensor, a DDS, a radio circuit, and an ASIC chip were integrated and embedded in a contact lens. For IOP sensing, a contact lens was designed with a diameter of 7.5 mm to fit rabbit's eyes, and for evaluation of IOP control, glaucoma was induced by injecting methylcellulose (MC) into an anterior chamber of the rabbit's eye or injecting α-chymotrypsin (α-chy) into a posterior chamber of the rabbit's eye. The glaucoma rabbits showed higher IOP than normal rabbits.

Thereafter, the contact lenses for IOP control were evaluated through the IOP monitoring and DDS.

A rabbit was secured in a cage and contact lenses were worn over its right (oculus dextrus (OD)) and left (oculus sinister (OS)) eyes. In the case of the IOP monitoring, initial IOP was measured with a commercially available tonometer, and IOP fluctuations were monitored after allowing the contact lens to be worn. Output codes were collected for 30 minutes at 5 minutes interval. After 30 minutes, the contact lens was removed from the rabbit's eye and the IOP was measured again with the tonometer.

For wireless monitoring and communication, a distance between a reception coil and a transmission coil embedded in the contact lens was maintained within 5 mm by aligning the two coils in parallel. The output codes were converted into IOP (mmHg) on the basis of a calibration standard curve for each rabbit.

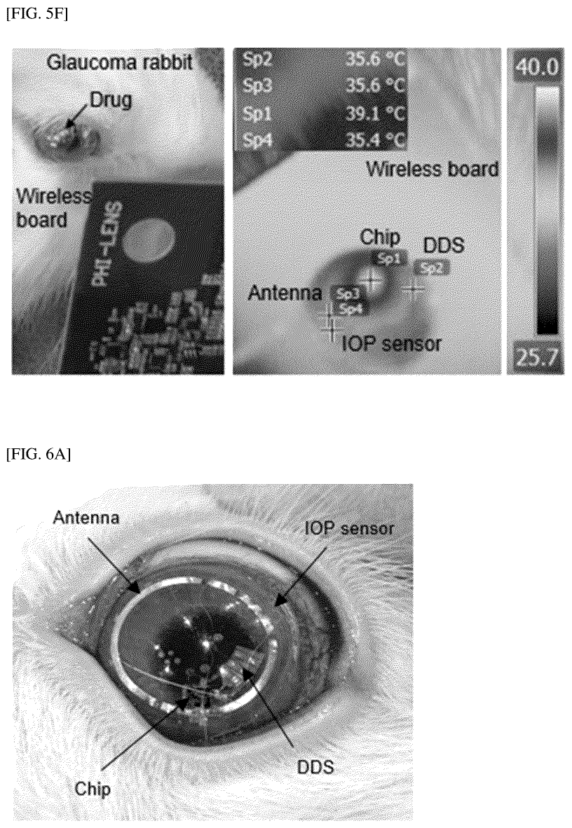

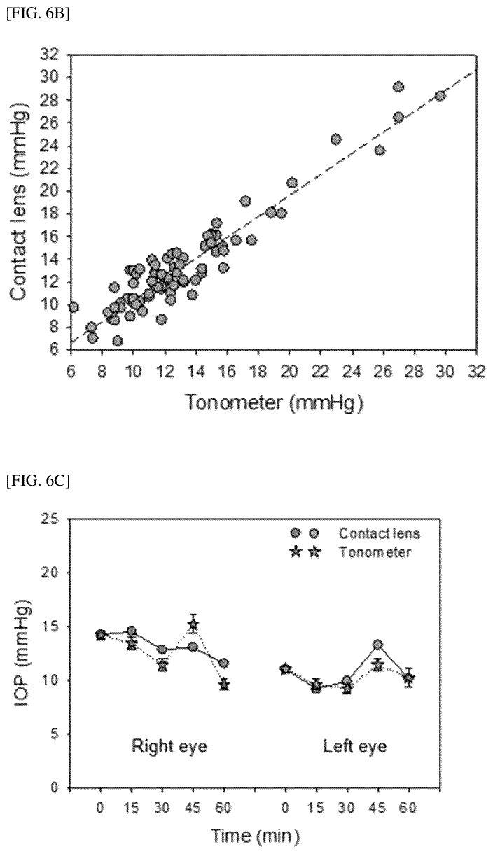

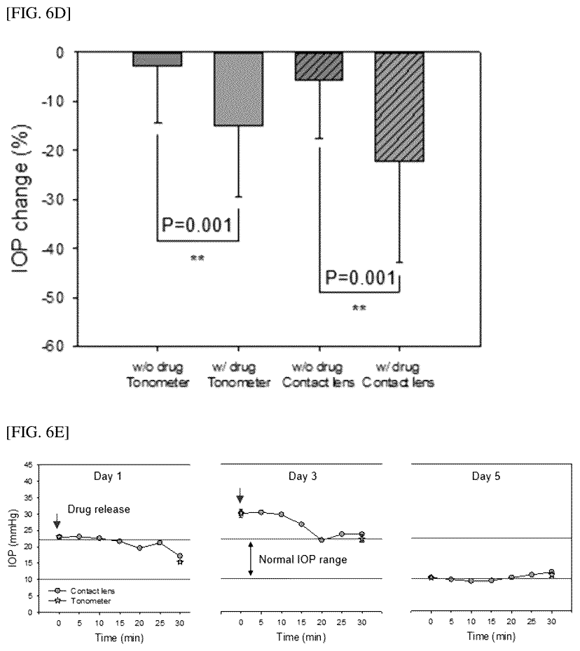

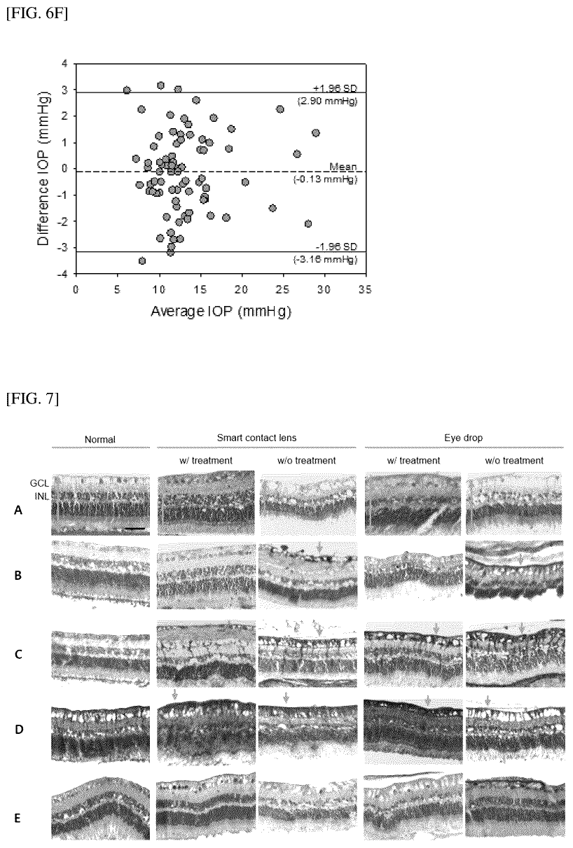

In the present invention, FIG. 6A shows a contact lens mounted on a rabbit's eye. An IOP sensor, a DDS, a radio circuit, and an ASIC chip were integrated and embedded in the contact lens.

FIG. 6B shows results of analysis of a correlation between the contact lens and a commercial tonometer, and FIG. 6F shows results of Bland-Altmann analysis after comparing the IOP measured by the commercial tonometer and a smart contact lens. Further, FIG. 6C shows continuous IOP measurement data of the contact lens. As shown in the drawings, it can be seen that, when IOP of a glaucoma-induced rabbit was measured using the contact lens and the commercial tonometer (rebound tonometer), there is a strong correlation between levels of IOP measured by the tonometer and the contact lens having a coefficient R2 of determination of 0.94. Further, it can be seen that an average difference in measured IOP between the two devices was about 0.13 mmHg, and a difference of up to 3.16 mmHg occurred. Therefore, it can be seen that the two devices have very high concordance.

Further, as a result of monitoring IOP profiles of the rabbit's OD and OS eyes for 60 minutes at 15-minute intervals using the contact lenses, it can be seen that the IOP profiles showed similar trends in both eyes.

FIG. 6D shows evaluation results of IOP lowering ability according to the presence or absence of timolol release in the contact lens.

It can be seen that, when IOP lowering ability of the drug released from the contact lens was measured using the contact lens and the commercial tonometer, a higher IOP lowering was observed in a drug-treated group than in a non-drug-treated group. Further, it can be seen that there is no statistical difference between the tonometer and the contact lens.

In particular, the contact lens rapidly reduced IOP during drug treatment, and the reduced IOP was maintained for 18 hours and returned to an initial IOP level after 24 hours.

Further, FIG. 6E shows experiment data of theranostic IOP measurement and adjustment analysis using a contact lens.

An IOP level may be controlled using the contact lens by monitoring the IOP and releasing timolol every other day for 5 days. Specifically, on the first day, since the IOP was in a high IOP range (22 mmHg or more), the timolol was released to lower the IOP and the IOP fell below a normal range. Even on day 3, the IOP was still above the normal range and the released timolol was able to reduce the IOP level near the normal range. On the last day, the IOP level was within the normal range and no timolol was released from the contact lenses.

Accordingly, it is possible to implement a contact lens system in which IOP monitoring and control are integrated together with an IOP sensor and a feedback system of a DDS for glaucoma treatment.

Experimental Example 8. In Vivo Therapeutic Effects of Contact Lens and Eye Drops

Therapeutic effects of contact lens and eye drops were evaluated by dividing glaucoma-induced rabbits and normal rabbits into 3 groups. In this case, glaucoma was induced by MC (M0512, viscosity 4,000 cP, Sigma-Aldrich) or α-chymotrypsin (C4129, Sigma-Aldrich).
A glaucoma rabbit group using eye drops (n=3, group 1),
A glaucoma rabbit group using contact lenses (n=3, group 2), and
A normal rabbit group serving as a control group (n=3, group 3).

Seven days after glaucoma induction, rabbits were given treatment in their right eyes and no treatment in their left eyes. Eye drops were administered to the right eyes every day for 16 days excluding weekends (a total of 12 days). The right eye was treated every other day for 16 days, excluding weekends, by receiving timolol (38 μg, 1 reservoir) from the contact lens (a total of 7 days of treatment).

### Experimental Example 9. Histopathological and Immunohistochemical Analysis

For preparation of 5 μm sections, the entire eyes fixed with formalin were embedded in paraffin. For histological evaluation, cut tissue was stained with hematoxylin and eosin (H & E; ABCAM, UK) and observed under a direct light microscope. Briefly, immunohistochemical detection involved antigen retrieval in 10 mM citrate buffer in a microwave, and endogenous peroxidase blocking with 1% hydrogen peroxide.

Tissues were incubated overnight at 4° C. with the following primary antibodies: glial fibrillary acidic protein (GFAP) (sc-51908, Santa Cruz); CD11b (ab8878, ABCAM); brain-derived neurotrophic factor (BDNF) (ab108619, ABCAM); and Brn3a (ab345230, ABCAM).

Next, a VECTASTAIN Elite ABC reagent for horseradish peroxidase (horse anti-mouse/rabbit IgG, Vector Laboratories, Burlingame, CA) was used for immunohistochemistry. Thereafter, the tissues were incubated and stained in a peroxidase substrate solution (ImmPACT 3,3′ diaminobenzidine (DAB) Substrate, Peroxidase, Vector Laboratories, Burlingame, CA) to the desired intensity, and then lightly counterstained with nuclear fast red (Abcam, UK). A dilution ratio for all antibodies listed above was 1:1,000.

FIG. 7 shows results of glaucoma-related biomarker analysis in a glaucoma-induced rabbit's retina using evaluation results of glaucoma therapeutic ability of a contact lens. Specifically, FIG. 7A shows retinal tissue analysis, FIG. 7B shows GFAP, FIG. 7C shows CD11b, FIG. 7D shows BDNF, and FIG. 7E shows expression results of a Brn3a biomarker.

The thickness of the retina was found to be similar to the normal thickness in two treated groups. However, in the case of the untreated group, the thickness of the retina was reduced.

Specifically, it can be seen that the structure of the retina was better maintained and the structures of a ganglion cell layer (GCL) and an inner nuclear layer (INL) were clearly observed in the treated group using the contact lens than in the untreated group.

The GFAP that is a marker associated with retinal damage was increased in the untreated group and was similar to that in the normal group in the treated group.

The CD11b that is an optic nerve damage marker was mainly exhibited in the glaucomatous retina and the untreated group, and the BDNF and brain-specific hmeobox/POU domain protein 3A (Brn3a), which are ganglion cell markers, were highly exhibited in the normal and treated groups.

Accordingly, glaucoma inhibition ability of contact lenses can be confirmed.

According to the present invention, gold hollow nanowires can be applied as a material for an intraocular pressure sensor having excellent safety and stability in vivo and excellent sensitivity.

Further, according to the present invention, a contact lens can monitor intraocular pressure in real time and release appropriate drug from a controlled drug delivery system according to a state of the intraocular pressure, thereby enabling personalized intraocular pressure adjustment.

What is claimed is:

1. A contact lens for measuring intraocular pressure or treating glaucoma in glaucoma patients, the contact lens comprising an intraocular pressure sensor and a drug reservoir,
wherein the intraocular pressure sensor includes gold hollow nanowires and measures a change in curvature of an eyeball caused by a change in intraocular pressure.

2. The contact lens of claim 1, wherein the contact lens is based on one or more selected from a group consisting of an elastomer such as a silicone elastomer, silicone hydrogel, and polymer hydrogel such as poly(2-hydroxyethyl methacrylate) (PHEMA), polyvinylpyrrolidone (PVP), poly(lactic acid-glycolic acid) (PLGA), or polyvinyl alcohol (PVA).

3. The contact lens of claim 1, wherein the intraocular pressure sensor and the drug reservoir are formed on a transparent substrate, and
the transparent substrate includes one or more selected from a group consisting of parylene C polydimethyloxane (PDMS), a silicone elastomer, polyethylene terephthalate (PET), and polyimide (PI).

4. The contact lens of claim 1, wherein the intraocular pressure sensor includes:
a gold hollow nanowire layer formed on the transparent substrate;
a D-poly(3,4-ethylenedioxythiophene) (D-PEDOT) layer formed on the gold hollow nanowire layer; and
a passivation layer formed on the D-PEDOT layer.

5. The contact lens of claim 4, wherein the passivation layer includes one or more selected from a group consisting of thermoplastic polyurethane (TPU), parylene C PDMS, a silicone elastomer, polyethylene PET, and PI.

6. The contact lens of claim 1, wherein the intraocular pressure sensor has a circular structure or a straight-line structure and entirely or partially surrounds a cornea of the eyeball.

7. The contact lens of claim 1, wherein the drug reservoir includes:
an electrode pattern containing gold formed on a portion of a surface of the transparent substrate; and
a drug well layer formed on the electrode pattern and including one or more drug wells formed to be inserted toward the outside,
holes are formed in the transparent substrate, and
the electrode pattern surrounds the holes.

8. The contact lens of claim 7, wherein drug contained in the drug well is drug capable of treating glaucoma and is made in a form of powder.

9. The contact lens of claim 1, further comprising a circular antenna configured to transmit or receive power and signals to or from the outside through induced current and electromagnetic resonance,
wherein the circular antenna is formed on the transparent substrate.

10. The contact lens of claim 1, further comprising an application specific integrated circuit (ASIC) chip,
wherein the ASIC chip is formed on the transparent substrate.

11. A method of manufacturing the contact lens for measuring intraocular pressure or treating glaucoma in glaucoma patients according to claim 1, the method comprising:
forming a water-soluble sacrificial layer on a handling substrate;
forming a transparent substrate on the sacrificial layer;
forming an intraocular pressure sensor and a drug reservoir on the transparent substrate; and
transferring the transparent substrate on which the intraocular pressure sensor and the drug reservoir are formed into the contact lens.

12. The method of claim 11, wherein the sacrificial layer includes one or more selected from a group consisting of polyvinyl alcohol (PVA) and dextran.

13. The method of claim 11, wherein the forming of the intraocular pressure sensor on the transparent substrate includes:
forming a mask material for patterning on the transparent substrate;
coating gold hollow nanowires on the transparent substrate on which the mask material is formed through a lift-off process and forming a gold hollow nanowire layer;
forming a D-poly(3,4-ethylenedioxythiophene) (D-PEDOT) layer on the gold hollow nanowire layer; and
forming a passivation layer on the D-PEDOT layer.

14. The method of claim 11, wherein the forming of the drug reservoir on the transparent substrate includes:
forming an electrode pattern containing gold on a portion of a surface of the transparent substrate; and
forming a drug well layer including one or more drug wells on the electrode pattern;
supporting a drug into the drug well; and
forming a protection layer on the drug.

15. The method of claim 14, wherein one or more holes are formed in the transparent substrate on which the drug reservoir is formed,
the electrode pattern surrounds the one or more holes, and
the one or more holes are formed before or after the electrode pattern is formed on the transparent substrate.

16. The method of claim 14, wherein the drug well layer including the drug well includes one or more selected from a group consisting of SU-8, polydimethyloxane (PDMS), a silicone elastomer, and polyurethane acrylate (PUA).

17. The method of claim 16, further comprising forming an antenna and an application specific integrated circuit (ASIC) chip on the transparent substrate.
